(19) United States
(12) Patent Application Publication
(10) Pub. No.: US 2018/0289871 A1
(43) Pub. Date: Oct. 11, 2018

(54) VACUUM ASSISTED DRESSINGS, SYSTEMS AND PUMPS FOR SURGICAL INCISIONS AND SEROMA MANAGEMENT

(71) Applicant: John Eric Player, Sterling, IL (US)

(72) Inventor: John Eric Player, Sterling, IL (US)

(21) Appl. No.: 15/945,859

(22) Filed: Apr. 5, 2018

Related U.S. Application Data
(60) Provisional application No. 62/483,072, filed on Apr. 7, 2017.

(51) Int. Cl. A61M 1/00; A61F 13/02

(52) U.S. Cl. CPC A61M 1/0088; A61F 13/0206; A61F 13/0216; A61M 1/0035; A61M 1/0072; A61F 13/0213

(57) ABSTRACT

A vacuum overdressing sealing a wound. The dressing has a layer of overdressing that is generally rectangular with a top side and a bottom side and two longer ends and two shorter ends. The overdressing layer has a port through which a vacuum can be pulled when the wound is sealed by the dressing. The port is located near one of the ends of the dressing.

### CROSS-REFERENCES TO RELATED APPLICATIONS

This application claims priority to U.S. Provisional Patent Application, Ser. No. 62/483,072 titled “Vacuum Assisted Dressings, Systems and Pumps For Surgical Incisions and Seroma Management”, filed Apr. 7, 2017, the disclosure of which is incorporated by reference herein in its entirety.

### FIELD OF THE INVENTION

The disclosure relates generally to devices and systems useful for treating incisions and managing seromas in patients. More specifically, the disclosed vacuum overdressing has a vacuum port near an end of the dressing and can be used to manage and treat seromas following a surgical procedure.

### SUMMARY

Devices, systems and methods to promote healing of incisions and treating seromas following surgical procedure are disclosed herein.

According to an embodiment, a new vacuum overdressing is disclosed with a first layer of generally rectangular-shaped overdressing material having a vacuum port near an end of the overdressing material.

Embodiments of the vacuum overdressing may include one or more layers of different dressing materials attached to the first layer of overdressing material.

In an embodiment, a sponge layer is attached to the first layer of overdressing material.

In another embodiment, a second layer of overdressing material is attached to the first layer of overdressing material.

In another embodiment, the first layer of overdressing material is a hydrocolloid dressing.

In another embodiment, the first layer of overdressing material is an adhesive film dressing.

### BRIEF DESCRIPTION OF THE DRAWINGS

The foregoing aspects and many of the advantages of the disclosed embodiments will become more readily appreciated by reference to the following detailed description, when taken in conjunction with the accompanying drawings, wherein:

FIGS. 1A-1D are schematic drawings of a prior art wound management system with a vacuum and bolster dressing.

FIG. 2 is a schematic cross-sectional view of FIG. 1D, taken along the line A-A;

FIG. 3 is a schematic drawing of a new wound management system.

FIG. 4 is a schematic drawing of another example of a new wound vacuum bolster dressing system.

FIGS. 5A-5C are schematic drawings of a new vacuum for a seroma management system, with a cap and seal.

FIGS. 6A-6C are schematic drawings of a new vacuum for a seroma management system, with a cap, seal and christmas tree connector with a one-way valve.

FIG. 7 is a schematic drawing of a new seroma management system with a vacuum that has a disposal port and a disposal biohazard bag for storing fluid.

FIG. 8 is a schematic drawing of a new seroma management system with a vacuum that has an interchangeable valve and disposal port.

FIGS. 9A-9B are schematic drawings of a new self-sealing over dressing system for seroma management.

FIG. 10 is a schematic cross section of the over dressing system shown in FIG. 9B.

FIG. 11 is a schematic drawing of a new seroma management system with an infusion port.

FIG. 12 is a schematic top view of a new well dressing for use in seroma management.

FIG. 13 is a schematic cross section of the new well dressing shown in FIG. 12.

### DETAILED DESCRIPTION

Complex surgical wounds are expensive and cause patients significant morbidity and, at times, death. Management of postoperative wounds and post-operative seromas encourage incision healing prior to the formation of complex wounds. Appropriate management of post-operative wounds after mesh placement may reduce the incidence of mesh infection. Mesh infection alone is costly and a source of morbidity and mortality. The present invention is particularly useful to manage high risk incisions before they become wounds and to promote healing of at-risk incisions.

Systems that applying a vacuum over a wound or incision to help drain fluid from the area to help promote healing and reduce the chance of infection are known. In order to apply known vacuum devices, a sealed area must be created in the desired area using various layers of dressing over the wound. Because various types of dressing are used and the whole area must be covered, creating of the sealed area is a time intensive process.

Known wound treatment systems that use negative pressure have a vacuum overlayer (i.e., a layer that has a port for connecting to a vacuum) that is square. Because they are not specifically shaped or designed for seroma management for surgical incisions, known vacuum overlayers impractical for post-operative incision treatment.

Known negative pressure systems that are marketed for use with incisions are “single-use” and do not allow a physician to monitor or inspect the incision or aspirate a seroma near an incision without removing and replacing the multiple layers of dressing that create a vacuum sealed area over the incision. Having a system that is specifically shaped for seroma management for incisions and that can reduce the time intensive procedure of replacing multiple layers of dressing will save time and cost.

High output seromas can output from between about 200 ml-500 ml of fluid in 24 hours. This type of volume will fill up the vacuum chambers of known portable vacuum pumps in less time than the vacuum treatment is desired. Removing and re-applying the multiple layers of dressing that created a sealed area over the incisions is a time intensive process for a surgeon or anyone else that treats wounds or seromas, e.g., another physician or a nurse. Having a seroma management system that can be used with high output seromas without the need to remove the sealed dressing to empty the vacuum chamber is desirable.

Finally, while managing the seroma under vacuum pressure, it can be desirable to treat or irrigate the area near the incision with saline or other medicated fluid from time to time. Having a system where the area can be rinsed without the need to remove the air sealed dressing will save time and cost.

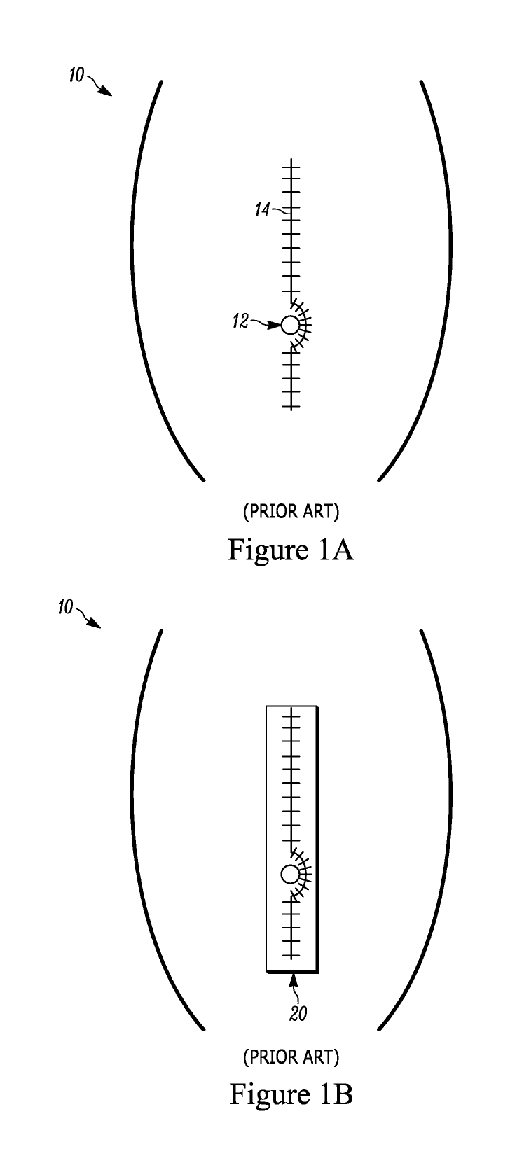

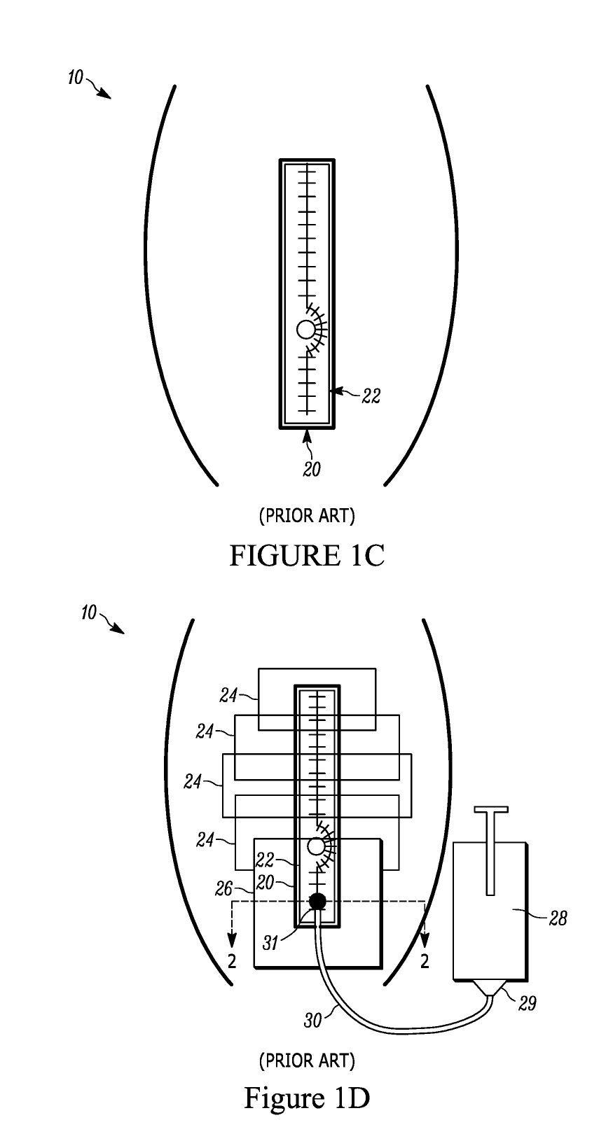

The discussion will now turn to FIGS. 1A-D, which are schematic diagrams showing an example of a known vacuum assisted seroma management system. FIG. 1A shows a patient's abdomen 10, with a typical abdominal incision 14 around an umbilicus or belly button 12. The incision 14 is generally straight, other than the curved section around the belly button 12. As shown, the incision 14 has been closed, e.g., by staples or sutures or other conventional means.

As shown in FIG. 1B, a layer of dressing 20 is applied over the closed incision 14. The dressing 20 helps protect the existing skin around the incision area. The dressing 20 can be a non-adhering or non-absorbent material designed to minimize wound adherence and prevent maceration. One example of such a dressing is sold under the trademark name Adaptic™ by Kinetic Concepts, Inc. Other types of non-adhering dressings are available and can be used.

As shown in FIG. 1C, a sponge 22 can be placed over the layer of nonadherent dressing 20. The sponge 22 is the media through which negative pressure is transmitted to the wound or incision. As is known in the art, the pore size of the sponge is selected to allow effective removal of exudate from the wound while maintaining negative pressure, i.e., suction. For example, the exudate of certain wounds tends to be thicker than for other wounds. If a proper pore size is not selected for the wound, the exudate can plug the sponge and prevent negative pressure from being applied.

As shown in FIG. 1D, additional layers of overdressings 24 can be added to seal the wound. Finally, a vacuum overdressing 26 that has an integral port 31 for attaching to a vacuum 28, can be placed over the incision in the area where the vacuum will be drawn. The overdressings 24 and 26 can be an adhesive film dressing or a hydrocolloid dressing and can be used in any combination, e.g., two hydrocolloid dressing layers 24 and 26, two adhesive film layers 24 and 26 or one layer of each material. The film dressing layer can be opaque or transparent. The hydrocolloid dressing layer can be any appropriate dressing, that is generally biodegradable and adheres to the skin, so no separate taping is needed. Examples of hydrocolloid dressings are sold under the trade names Exuderm, Comfeel Plus, Duoderm, Granuflex, Ultec, and 3M Tegaderm Hydrocolloid. Examples of adhesive film dressings are sold under the trade names Opsite and Tegaderm.

As shown in FIG. 1D, prior art vacuum overdressings 26 have a square shape and the integral suction port 31 is located in the center of the dressing 26. A tube 30 through which a negative pressure is created is connected to the vacuum port 31. The other end of the tube 30 is connected to a one-way valve 29 that is integrally formed with the vacuum 28. Generally, a one-way valve has one opening to allow fluid to enter and another opening to allow fluid to leave. There are a variety of one-way valves that are known and could be suitable for use. Here, the direction of fluid flow is from the tube 30 into the vacuum 28.

The vacuum 28 creates negative pressure at a desired pressure. For example, standard pressures in would care range from about −100 mmHg to −200 mmHg, depending on the size of the incision or desired negative vacuum. Often, the desired initial negative pressure is about −125 mmHG. Preferably the pump is small enough to be portable so that the patient can use it for up to several days, even when the patient is mobile. Known disposable pumps create a fairly consistent pressure of around −125 mmHg. The vacuum 28 may be a manual pump. Alternatively, a battery powered pump device may also be used. A reservoir or container (not shown) for collecting the fluid is also integral with the vacuum 28. For known disposable vacuums, the pump is the container. When a negative pressure is applied by the vacuum, fluid from the seroma may be drained from the port in the dressing, through the tube to the collection reservoir in the pump 28.

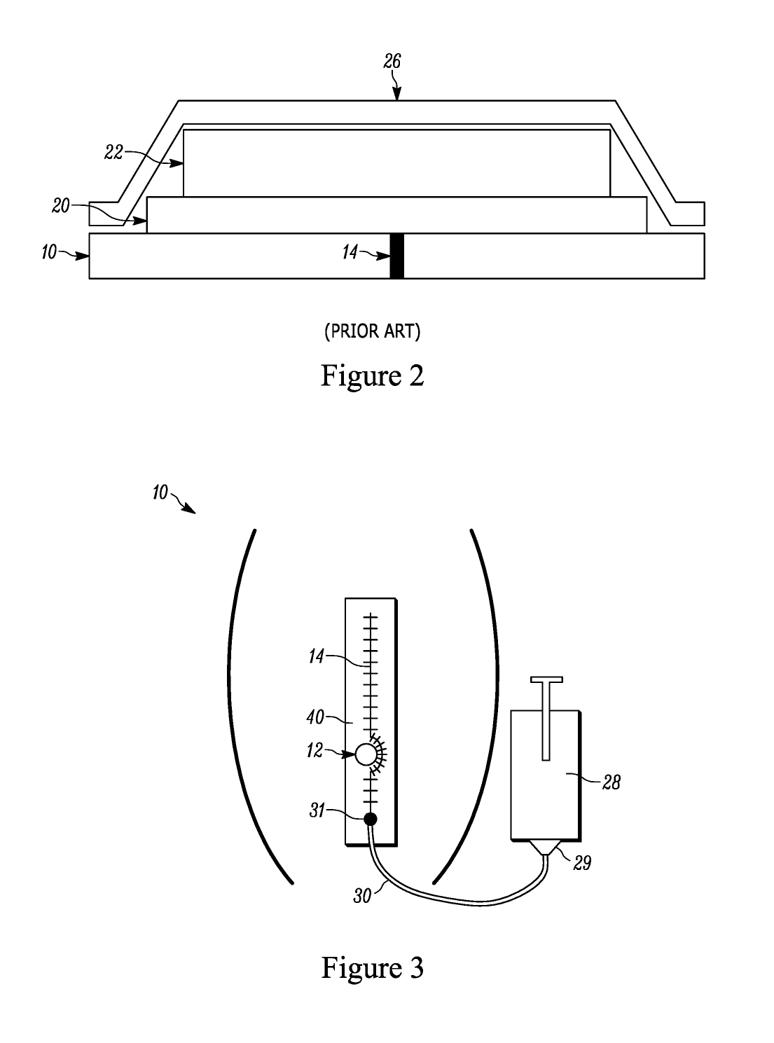

FIG. 2 is a cross sectional view of FIG. 1D, taken through the line 2-2. An over dressing layer 26 is shown applied over the sponge 22 and non-absorbent dressing 20. The overdressing layer 26 forms an airtight sealed area over the wound 14, dressing 20 and sponge 22, which allows the vacuum pump to create negative pressure on the wound area through the port 31.

As shown in FIG. 3, a new vacuum overdressing 40 is provided. The dressing 40 is generally rectangular-shaped to follow the shape of the incision 14. It may be made of a material similar to that of overdressing 26. In addition, the overdressing 40 has a vacuum port 31 located near an end or edge of the overdressing 40. The location of the port 31 near the end of dressing 40, allows the port 31 to be located near the lower end of the incision body when the dressing 40 is applied to a patient. Placement of the vacuum port 31 near the lowest part of the incision 14 assists in removal of fluid, as seroma fluid is most likely to settle near the lowest location.

In addition, the shape of the overdressing 40 allows a single layer of overdressing 40 to be used to seal an area around the incision 14. This helps reduce the time and effort of applying several other layers to seal the incision (e.g., layers 24 in FIG. 1D) prior to applying the vacuum over dressing. The overdressing 40 can be placed over other layers, such as a sponge and non-absorbent dressing (similar to those described in FIGS. 1 and 2), that may be needed to help protect the wound and facilitate vacuum flow.

However, because the shape of the vacuum overdressing layer 40 covers the entire incision, other layers may be formed integrally with over dressing layer 40 to help protect or treat the incision. For example, a sponge layer (similar to layer 22 in FIG. 1C) can be adhesively attached underneath or integrally formed with the layer 40. Alternately, a non-absorbent dressing (similar to the dressing 20 in FIG. 1B) can be attached underneath or integrally formed with the sponge layer or layer 40. In another example, a non-absorbent dressing (similar to the overdressing 20 in FIG. 1B) can be attached to the sponge layer (similar to layer 22 in FIG. 1C) or dressing 20.

Other dressing layers can also be provided. For example, a second overdressing layer may be positioned between the sponge 22 and the layer 40. Additionally, a second non-absorbent dressing layer may be applied over the non-absorbent dressing layer that is applied over the wound.

As described herein, the vacuum 28 may be powered mechanically, by batteries or by AC electricity. In addition, the vacuum 28 may be disposable after a single use or a number of cycles for a given patient. The pump can also be adjustable to create the desired level of vacuum, ideally between about −100 and −200 mmHg, although lower or high vacuum levels may be desired for certain treatments.

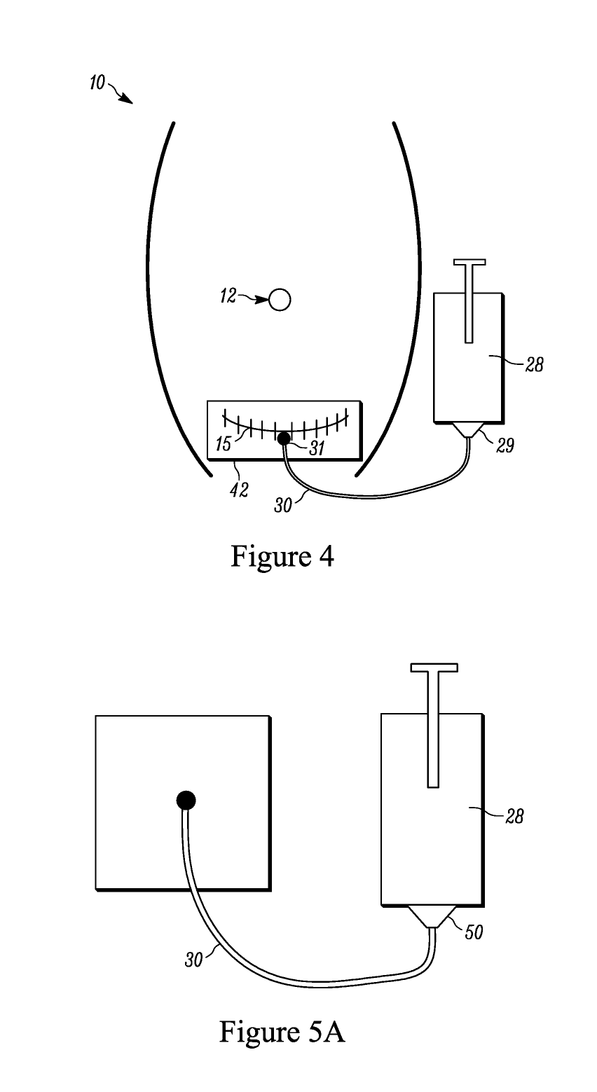

In FIG. 4, another example of a vacuum overdressing 42 is shown that can be used with a different type of incision 15, such as a Pfannenstiel incision. The overdressing 42 is generally rectangular and has an integral port 31 located near the center of the dressing 42. It is made of material similar to overdressing 26 and 40. This dressing shape and port configuration allows the dressing 42 to cover the entire incision, creating a single layer seal over the entire incision without the need for other additional dressings. In addition, the port 31 is positioned so that when the overdressing 42 is applied to a Pfannenstiel incision, it will be positioned in a location where seroma fluid is most likely to settle, i.e., near the lowest point of the incision. Thus, when the vacuum 28 is attached to the port 31, drainage of the seroma fluid is improved due to the location of the port 31 in the dressing 42.

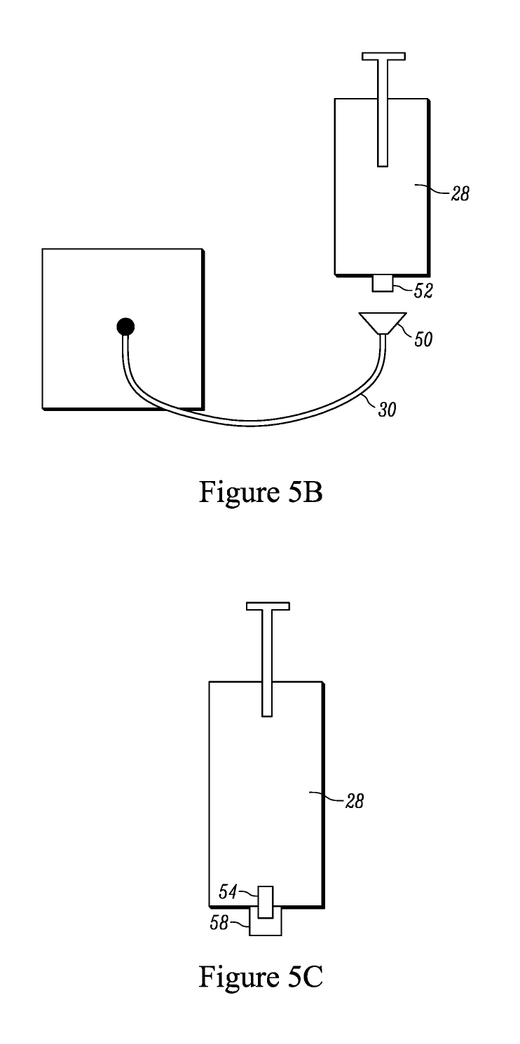

FIGS. 5A-5C are schematic drawings of a new wound vacuum pump 28 for seroma management that has a cap 58 for sealing the vacuum after it is full. As shown in FIG. 5A, the vacuum pump 28 has a one-way valve 50. Like the one-way, valve 29 of FIG. 1, the one-way valve 50 is removable from the vacuum. As shown in FIG. 5B, the valve 50 is attached to the vacuum on an extension or post 52 at the bottom of the vacuum. However, the valve can be attached by any other suitable method or structure that allows it to be sealed when attached, but allows it to be detached from the body of the pump 28. The vacuum 28 has a hole (not shown) that matches up with a valve opening to allow fluid to flow through the valve 50 into the vacuum 28. When the vacuum is full, the one-way valve 50 can be detached from the vacuum 28, as shown in FIG. 5B.

As shown in FIG. 5C, in order to seal the vacuum 28 and prevent fluid from escaping from the vacuum 28 or vacuum chamber, a biohazard seal 54 (e.g., plastic seal) can be placed around the post or hole in vacuum 28 wall. A cap 58 can also be placed onto the extension or post 52 at the bottom of the vacuum, and over the seal 54. Once the vacuum 28 is sealed by the seal 54 and cap 58, it can be transported without leaking any fluid. It can then be emptied in a safe place or disposed of with the seroma fluid safely sealed inside the vacuum 28. The cap may be the same general size and shape of the one-way valve, except that it does not have a channel, so fluid cannot flow through the cap.

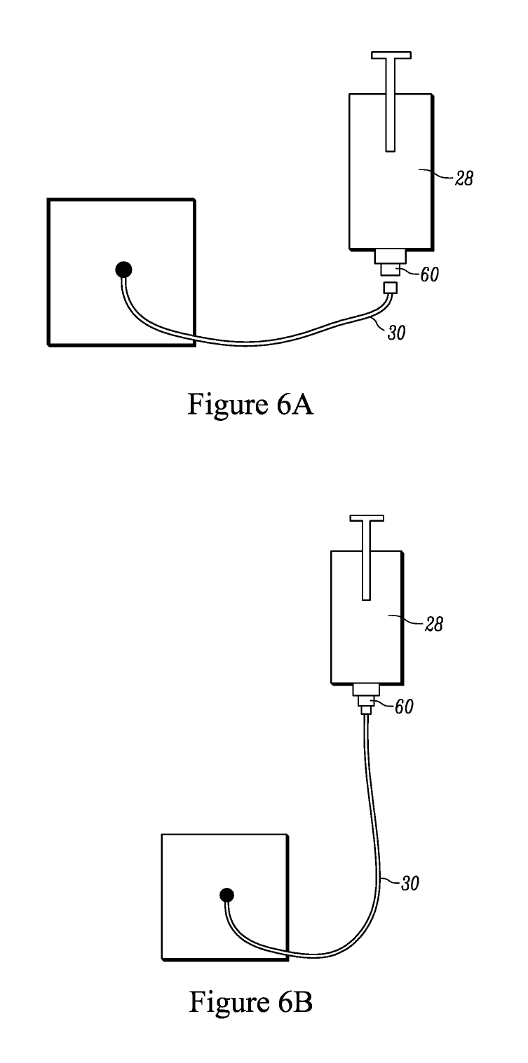

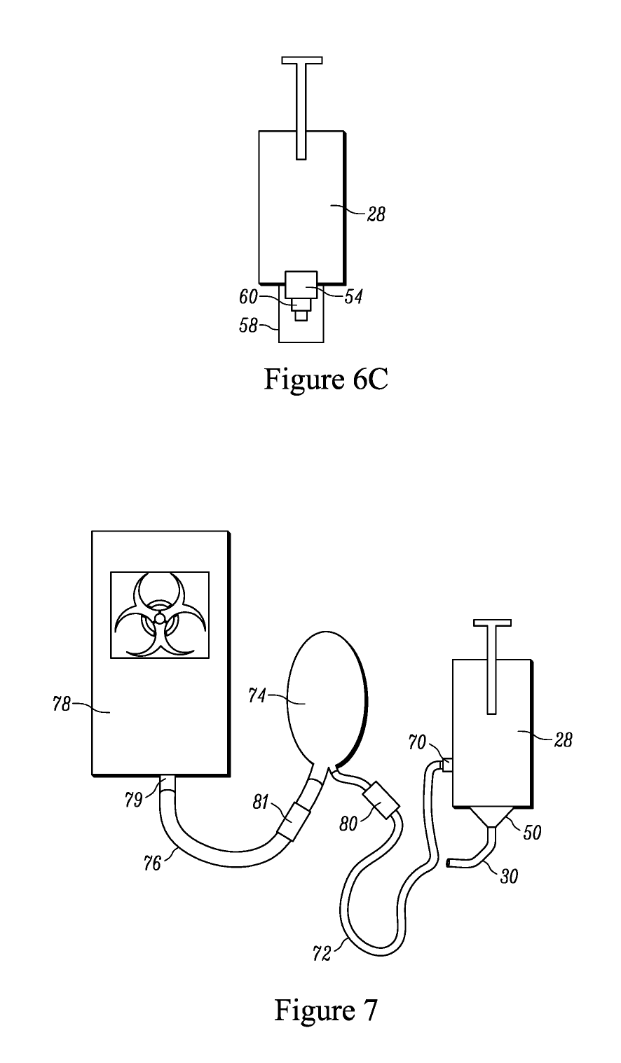

FIGS. 6A-6C are schematic drawings of another new cappable vacuum for a seroma management system, with a cap 58, seal 54 and christmas tree-shaped one-way valve 60. As shown in FIG. 6A, the one-way valve can be a christmas tree-shaped connector valve 60 which connects to the tube 30. The valve 60 can be fixed to the vacuum 28 or removable (in a manner similar to the valve 50 described above.) As shown in FIG. 6B, the tube 30 can be attached by press fitting it over the end of the connector 60 until it is snug. This deforms the end of the plastic tubing 30. When the vacuum 28 is full, the tubing 30 can be pulled off the end of the connector 60, and the vacuum can be sealed, as shown in FIG. 6C, to seal fluid inside the vacuum 2. To seal the fluid inside the pump, a cap 58 is press fit or friction fit over the christmas tree-shaped connector valve. Once the vacuum 28 is sealed, it can be transported without leakage and then either drained in a safe place or disposed of in a safe manner with the fluid inside the vacuum 28.

The deformed end of the tube 30 can be cut off to get a new clean, undeformed edge of the tube 30. The new edge can then be reattached to a valve 60 on a new pump 28 (or the same pump if it is drained and being re-used) so that the dressing does not have to be removed. Clipping the deformed end and using a new clean end helps maintain a good seal and allows the sealed overdressing to remain in place, thus avoiding the time intensive process of re-dressing and re-sealing the incision.

FIG. 7 is a schematic drawing of a new seroma management system with a vacuum 28 that has a disposal port 70 and a disposal biohazard bag 78 for storing fluid. The pump can have a one-way valve (similar to any of the valves described above). A disposal port 70 is provided in a wall of the vacuum to allow the vacuum to be drained and re-used.

When the vacuum is in use, the port 70 is sealed (not shown), e.g., with a cap, plug or similar structure, to prevent fluid from escaping from the pump 28. To drain fluid from the pump, the port 70 can be unsealed and connected to a biohazard bag or other receptacle, as shown in FIG. 7. As shown in FIG. 7, one end of tubing 72 can be connected to the port 70. The other end of the tubing 70 can be connected to a silicone bulb pump 74. The bulb can also be connected to an end of a second silicone tubing 76. The other end of the tubing 76 is connected, via a port 79, to a disposable biohazard bag 78. However, any suitable container for collecting the fluid may be used. Alternatively, a container need not be connected, by fluid can be allowed to flow into another container or proper disposal area.

Preferably, the tubing 72 and 76 is silicone tubing. Also, preferably tubing 72 is a smaller diameter than tubing 76. Pinch clamps 80 and 81 can also be provided to seal the tubing 72 and/or 76 when they are clamped, as desired to prevent fluid from flowing to or from the tubing or other components of the system.

To pull fluid from the vacuum 28 and into the bulb pump 74, clamp 80 is not clamped on (and does not seal) tube 72 and clamp 81 is clamped on (and seals) tube 76. In this configuration, when the bulb pump 74 is pumped, fluid flows from the vacuum 28, through tube 72 and into bulb pump 74.

To pump fluid into the receptacle 78, the clamps are changed so that clamp 80 is clamped on (and seals tube 72) and clamp 81 is not clamped on (and does not seal) tube 76. In this configuration, when the bulb 74 is pumped, fluid from the bulb 74 will flow through the tube 76 and into the container 78.

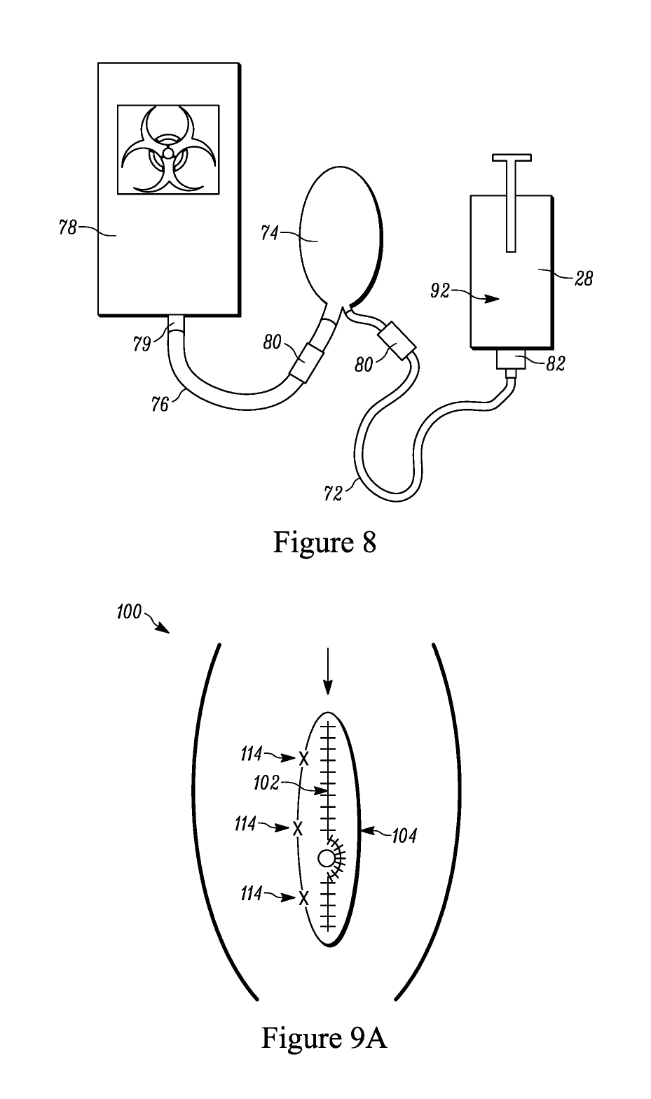

FIG. 8 is a schematic drawing of another example of new seroma management system with a vacuum that has an interchangeable valve and disposal port. This vacuum can have a removable one-way valve (like the one shown in FIG. 5), that is removed when the vacuum is full or when it is desired to be drained of fluid. A fitment 82 without a valve can be inserted onto the vacuum 28. The vacuum 28 can then be drained through tubes 72 and 76 using bulb pump 74, by different configurations of the clamps on tubes 72 and 74, as described above in FIG. 7.

In addition, to maintain the contents of the vacuum in a liquid state to facilitate evacuation into the bag, citrate 92 may be added to the chamber of the vacuum to keep it from coagulating. In addition, a congealer powder can be added to the biohazard bag to help convert the liquid to a congealed solid waste to reduce leakage and make it easier to seal.

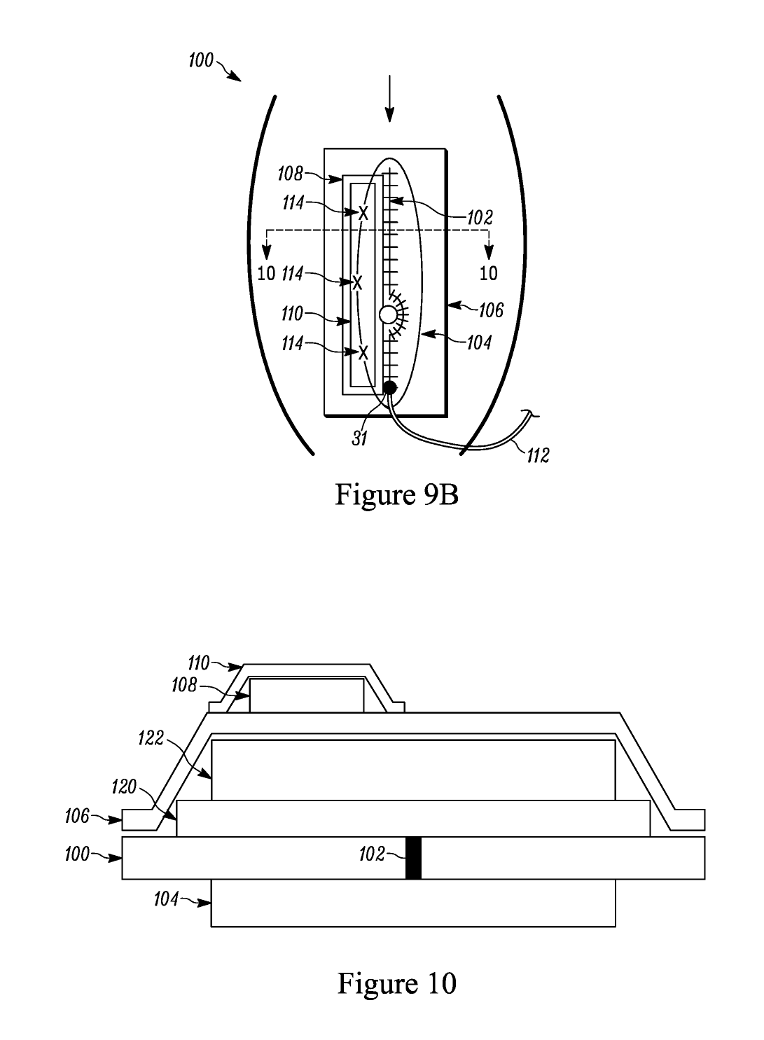

FIGS. 9A-9B are schematic drawings of a self-sealing over dressing system for seroma management. As shown in FIG. 9A, an incision 102 has been closed, e.g., by staples or sutures or other conventional mean, in an abdomen 100 may have a seroma built up underneath it in the region designated by the area 104. Three locations where it may be desired to insert a needle to drain the seroma are designated by “x” 114.

As shown in FIG. 9B, a generally rectangular overdressing 106 (similar to the overdressing 40 described above in FIG. 3, e.g., hydrocolloid layer or film layer) with a port 31 is applied over the incision 102. Also as shown in FIG. 10, other layers may be applied between the skin 100 and the overdressing 106 and the layers may be integrally formed with the overdressing layer 106. When the overdressing 106 is applied to the skin, it creates a sealed area underneath the dressing 106 and around the incision 102. A vacuum can be formed in the area under the dressing 106. A layer of self-sealing overdressing 108 may be applied on top of the overdressing 106. The self-sealing overdressing 108 can be applied so that it covers each of the locations 114 where the needle will be inserted to drain the seroma. Another layer of overdressing 110 (similar to the overdressing 40 described above 106, i.e., hydrocolloid layer or film layer) is applied over the self-sealing overdressing layer 108. The layers 108 and 110 can have a layer of adhesive on the bottom of each layer to allow them to be applied as separate layers on top of the vacuum overdressing layer 106. Alternatively, layers 108 and 110 can be integrally formed with, e.g., by lamination, with layer 106 and applied to a patient as a single unit. As shown, the layers 106, 108 and 110 are directly adjacent to each other, however, additional layers may be present between the layers 106, 108 and 110.

After the overdressing layers 106, 108 and 110 are in place, the seroma can be drained with a needle at the three “x” locations 114 by inserting the needled through the layers 106, 108 and 110. The needle will puncture the seal created by the vacuum layer 106. However, after the seroma is drained and the needle is remove, layers 108 and 110 will re-seal, so that a seal is preserved under the entire dressing (layers 106, 108 and 110) and a vacuum can still be pulled by the pump through tube 112. This allows for aspiration of a seroma, without the time-consuming task of removing and re-applying one or more layers of vacuum dressing 106 in an airtight manner.

The self-sealing overdressing layer 108 is made of a moldable adhesive, that is sticky when warmed to body temperature, such as an adhesive layer sold under the trade name Eakin Cohesive. The seroma 104 is aspirated by inserting a needle through layers 110, 108, 106, 122 and 120, as well as the skin 100. After the seroma 104 has been aspirated, the needle is removed from all of the layers, and pressure can be applied to layer 108 to obliterate and seal the needle tract. Preferably the layer 110 is a clear adhesive layer in order to reinforce the defect created by needle tract over the site and to prevent the moldable layer 108 from becoming adherent to the patient's clothing or other objects in the environment, including dirt and debris.

FIG. 10 is a schematic cross section of the over dressing system shown in FIG. 9B generally taken across the line 10-10. Although not seen in FIG. 9, a layer of non-absorbent dressing 120 is present on the skin over the incision 102. A layer of foam 122 is located between the non-absorbent layer dressing layer 120 (similar to the foam and non-absorbent layers 20 and 22 described above) and the vacuum seal dressing layer 106. The vacuum seal dressing layer 106 is placed over the incision, and over layers 120 and 122. The layers 120, 122, 106, 108 and 110, may be separate layers, applied separately and bonded to each adjoining layer by adhesive. Alternately, the self-sealing dressing can be manufactured as a single unit, e.g., by lamination or use of adhesive layers. Additional layers can also be provided above or below each of the layers shown.

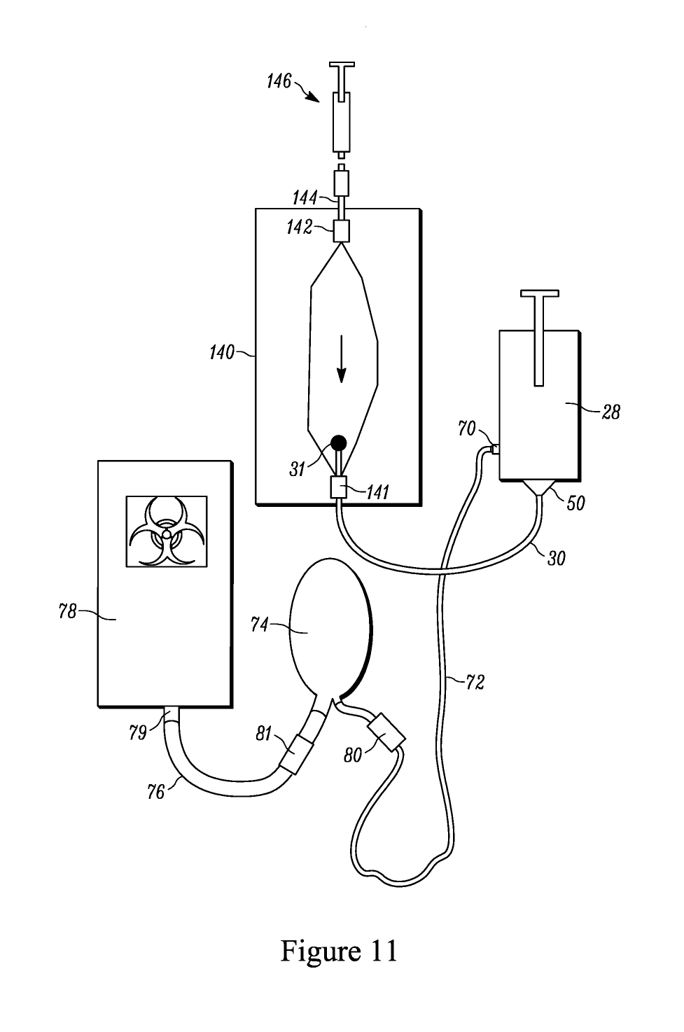

FIG. 11 is a schematic drawing of a new seroma management system with an infusion port.

A vacuum seal dressing layer 140 (similar to the vacuum seal layers 40 and 106 described above) is placed over an incision to form a sealed area around an incision. Other layers may be below the vacuum seal layer, e.g., a foam sponge layer (such as layer 122 described above) and a dressing layer (such as layer 120 described above).

Similar to the system described in FIGS. 3 and 7, the vacuum seal layer 140 has a port 31, which is attached to a tube 30. A vacuum 28 can draw a vacuum through a one-way valve 50 and tube 30 to drain a seroma.

In addition, and similar to the system described in FIG. 7, the pump can have a one-way valve 50 and a disposal port 70.

When the vacuum is in use, the port 70 is sealed (not shown), e.g., with a cap, plug or similar structure, to prevent fluid from escaping from the pump 28. To drain fluid from the pump, the port 70 can be unsealed and connected to a biohazard bag or other receptacle. As described above, one end of tubing 72 can be connected to the port 70. The other end of the tubing 70 can be connected to a silicone bulb pump 74. The bulb can also be connected to an end of a second silicone tubing 76. The other end of the tubing 76 is connected, via a port 79, to a disposable biohazard bag 78. However any suitable container for collecting the fluid may be used. Alternatively, a container need not be connected, by fluid can be allowed to flow into another container or proper disposal area.

Preferably, the tubing 72 and 76 is silicone tubing. Also, preferably tubing 72 is a smaller diameter than tubing 76. Pinch clamps 80 and 81 can also be provided to seal the tubing 72 and/or 76 when they are clamped, as desired to prevent fluid from flowing to or from the tubing or other components of the system.

To pull fluid from the vacuum 28 and into the bulb pump 74, clamp 80 is not clamped on (and does not seal) tube 72 and clamp 81 is clamped on (and seals) tube 76. In this configuration, when the bulb pump 74 is pumped, fluid flows from the vacuum 28, through tube 72 and into bulb pump 74.

To pump fluid into the receptacle 78, the clamps are changed so that clamp 80 is clamped on (and seals tube 72) and clamp 81 is not clamped on (and does not seal) tube 76. In this configuration, when the bulb 74 is pumped, fluid from the bulb 74 will flow through the tube 76 and into the container 78.

In addition, in this system, the vacuum seal layer 140 can have a second port 142. The port can be used to inject fluid into the area over the incision to clean it. Prior to injecting the fluid, a pinch clamp can be used to seal the tube 30 to prevent the fluid from flowing out of the sealed area under the layer 140. A silicone tube 142 is attached to the port 10, which can be connected to a saline flush by way of an IV-lock device (or other connector) to allow the saline to be injected into the area around the incision. After the saline has been injected, the pinch clamp 142 can be applied to tube 144 to keep the saline in the area around the incision for a desired period of time. When the saline is desired to be removed, the pinch clamp 141 can be removed and the vacuum can apply negative pressure to drain the saline and other fluid from the area into the vacuum 28.

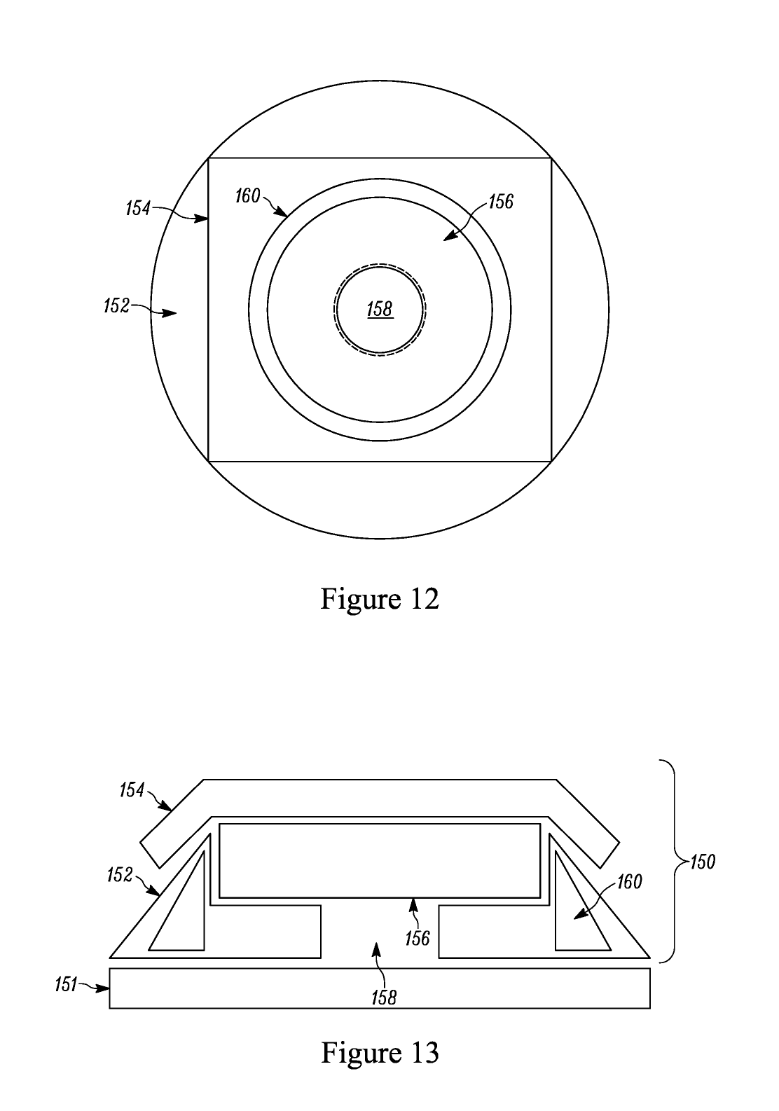

FIGS. 12 and 13 show a “well” dressing 150 that can be used in connection with draining a seroma. FIG. 12 is a schematic top view of the “well dressing and FIG. 13 is a cross sectional view of the “well” dressing. This “well” is used on top of the sealed dressing placed over an incision, such as the sealed dressings described in FIGS. 3, 4 and 9. As described above, from time to time it is desirable to drain a seroma using a needle that is inserted through the sealed dressing layers. The “well” dressing can be sealed to allow the original dressing to remain in place after a needle has been used to drain the seroma through the dressing.

When a vacuum sealed dressing is applied to an incision, or even after it is applied, the best location to drain a seroma with a needle, i.e., the location that will remove the most fluid, is not always known. The “well” 150 can be used over a vacuum dressing layer or area of a vacuum dressing layer that is not self-sealing and allows the needle to be inserted in a desired point on the sealed dressing, and, if needed, at multiple points on the sealed dressing.

The “well” 150 can be placed on top of a vacuum dressing layer 151 (FIG. 13) similar to those descried above in FIGS. 3-11, e.g., dressing 40 (FIG. 3) or 42 (FIG. 4). The “well” 150 consists of a layer of hydrocolloid dressing material 152. The layer 152 has a cut out section and a hole 158. A plastic ring 160 can also be formed in the layer 152 or around the hole 158 to provide support for the layer of dressing 152.

A needle can be inserted through the hole 158 and vacuum layer dressing 151 to drain the seroma. After the seroma has been drained, the needle is removed, and a layer of moldable self-sealing material 156 (similar to the self-sealing material layer 108 described above) can be placed inside the cut out. Then, a layer of film over dressing 154 is placed over the layer 152 and 154 to form a seal again. Pressure can be applied to the moldable self-sealing layer 156 prior to or after applying the film layer 154 in order to seal the hole 158. The ring provides support such that when pressure is applied to the layer 156, the layer 156 effectively seals the hole and the space in the cut-out.

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Not all steps listed in the various figures need be carried out in the specific order described.

The invention claimed is:

1. A vacuum overdressing for sealing a wound comprising:
a layer of overdressing;
wherein the overdressing layer is generally rectangular having a top side and a bottom side, and defined by two longer ends and two shorter ends,
and further wherein the overdressing layer has a port through which a vacuum can be pulled when the wound is sealed by the dressing;
said port being located near one of the shorter ends of the dressing.

2. The vacuum overdressing of claim 1, further comprising:
a sponge layer attached to the bottom side of the overdressing layer.

3. The vacuum overdressing of claim 2, wherein the sponge layer is integrally formed with the layer of overdressing.

4. The vacuum overdressing of claim 2, wherein the sponge layer is attached to the layer of overdressing by an adhesive.

5. The vacuum overdressing of claim 2, further comprising a layer of non-absorbent dressing attached to the sponge layer.

6. The vacuum overdressing of claim 1, further comprising a layer of non-absorbent dressing attached to the layer of overdressing.

7. The vacuum overdressing of claim 1 wherein the layer of overdressing is an adhesive film dressing.

8. The vacuum overdressing of claim 1 wherein the layer of overdressing is a hydrocolloid dressing.

9. The vacuum overdressing of claim 2, further comprising a second layer of overdressing between the first layer of overdressing and the sponge layer.

10. A vacuum overdressing for sealing a wound comprising:
a layer of overdressing;
wherein the overdressing layer is generally rectangular having a top side and a bottom side, and defined by two longer ends and two shorter ends,
and further wherein the overdressing layer has a port through which a vacuum can be pulled when the wound is sealed by the dressing;
said port being located near one of the longer ends of the dressing.

11. The vacuum overdressing of claim 10, further comprising:
a sponge layer attached to the bottom side of the overdressing layer.

12. The vacuum overdressing of claim 11, wherein the sponge layer is integrally formed with the layer of overdressing.

13. The vacuum overdressing of claim 11, wherein the sponge layer is attached to the layer of overdressing by an adhesive.

14. The vacuum overdressing of claim 11, further comprising a layer of non-absorbent dressing attached to the sponge layer.

15. The vacuum overdressing of claim 10, further comprising a layer of non-absorbent dressing attached to the layer of overdressing.

16. The vacuum overdressing of claim 10 wherein the layer of overdressing is an adhesive film dressing.

17. The vacuum overdressing of claim 10 wherein the layer of overdressing is a hydrocolloid dressing.

18. The vacuum overdressing of claim 11, further comprising a second layer of overdressing between the first layer of overdressing and the sponge layer.
